(19) United States
(12) Patent Application Publication
(10) Pub. No.: US 2025/0378228 A1
(43) Pub. Date: Dec. 11, 2025

(54) SIMULATION METHOD FOR SELECTING OPTIMAL COMPOSITION RATIO OF OXIDE SEMICONDUCTOR, AND ELECTRONIC DEVICE INCLUDING THE OXIDE SEMICONDUCTOR

(71) Applicant: Samsung Display Co., LTD., Yongin-si (KR)

(72) Inventors: Young Mi CHO, Yongin-si (KR); Sang Gu LEE, Yongin-si (KR); Ji Young LEE, Yongin-si (KR)

(73) Assignee: Samsung Display Co., LTD., Yongin-si (KR)

(21) Appl. No.: 19/081,600

(22) Filed: Mar. 17, 2025

(30) Foreign Application Priority Data
Jun. 5, 2024 (KR) 10-2024-0073615

(51) Int. Cl. G06F 30/20; G06F 113/26; G06F 119/02; H10D 62/80; H10D 62/86; H10D 62/862

(52) U.S. Cl. CPC G06F 30/20; H10D 62/8603; H10D 62/862; H10D 62/875; G06F 2113/26; G06F 2119/02

(57) ABSTRACT

The disclosure relates to a simulation method for selecting an optimal composition ratio of an oxide semiconductor. The oxide semiconductor includes at least two elements selected from the group consisting of indium (In), gallium (Ga), zinc (Zn), tin (Sn), silver (Ag), aluminum (Al), cadmium (Cd), magnesium (Mg), antimony (Sb), silicon (Si), titanium (Ti), and zirconium (Zr); oxygen (O); and inevitable impurities. The simulation method includes setting a simulation target composition ratio set including various composition ratios of elements constituting the oxide semiconductor, checking whether the oxide semiconductor satisfies Formulas 1, 2, and 3 for each of the various composition ratios included in the simulation target composition ratio set, and selecting a composition ratio satisfying Formulas 1, 2, and 3 as an optimal composition ratio. Formulas 1, 2, and 3 may be the same as described in the specification.

### CROSS-REFERENCE TO RELATED APPLICATION(S)

This application claims priority under 35 U.S.C. § 119 to and benefits from Korean Patent Application No. 10-2024-0073615 filed on Jun. 5, 2024 in the Korean Intellectual Property Office, the entire contents of which are incorporated herein by reference.

### BACKGROUND

### 1. Technical Field

The disclosure relates to a simulation method for selecting an optimal composition ratio of an oxide semiconductor, and an electronic device including the oxide semiconductor.

### 2. Description of the Related Art

Thin film transistors are used in various electronic devices, such as flat panel displays. For example, thin film transistors are used as switching elements or driving elements in flat panel display devices, such as liquid crystal displays, organic light emitting diode (LED) displays, and micro LED display devices.

A thin film transistor includes a gate electrode connected to a scan line that transmits a scan signal, a source electrode connected to a data line that transmits a signal to be applied to a pixel electrode, a drain electrode facing the source electrode, and a semiconductor electrically connected to the source electrode and the drain electrode.

Among these, the semiconductor may play an important role in determining characteristics of the thin film transistor. Recently, research has been conducted on thin film transistors using oxide semiconductors, which have higher carrier mobility, higher ON/OFF ratio, lower cost, and higher uniformity than amorphous silicon.

In such cases, characteristics of thin film transistors may be determined by composition ratios of oxide semiconductors.

### SUMMARY

The disclosure provides a simulation method for selecting an optimal composition ratio of an oxide semiconductor.

The simulation method in accordance with embodiments of the disclosure is a simulation method for selecting an optimal composition ratio of an oxide semiconductor. The oxide semiconductor may include at least two elements selected from the group consisting of indium (In), gallium (Ga), zinc (Zn), tin (Sn), silver (Ag), aluminum (Al), cadmium (Cd), magnesium (Mg), antimony (Sb), silicon (Si), titanium (Ti), and zirconium (Zr); oxygen (O); and inevitable impurities. The simulation method may include setting a simulation target composition ratio set including various composition ratios of elements constituting the oxide semiconductor; checking whether the oxide semiconductor satisfies Formulas 1, 2, and 3 below for each of the various composition ratios included in the simulation target composition ratio set; and selecting a composition ratio satisfying Formulas 1, 2, and 3 below as an optimal composition ratio:

$$Ef ( Con - IGZO ) - 0.1 eV < Ef < Ef ( Con - IGZO ) + 0.3 eV \qquad [ Formula 1 ]$$

$$2 eV < E g < 3.5 eV \qquad [ Formula 2 ]$$

$$100 \cdot {( ISWOc )}^{- 2} \cdot exp ( Ef ) > 3 \qquad [ Formula 3 ]$$

wherein, in Formulas 1, 2, and 3, Ef may be formation energy of the oxide semiconductor calculated based on density functional theory (DFT), Eg may be bandgap energy of the oxide semiconductor calculated based on the DFT, ISWOc may be an Inverse State Weighted Overlap of Conduction band parameter of the oxide semiconductor, Ef (Con-IGZO) may be formation energy of conventional-IGZO oxide, the conventional-IGZO oxide includes indium (In), gallium (Ga), zinc (Zn), and oxygen (O), indium (In): gallium (Ga): zinc (Zn) in the conventional-IGZO oxide satisfies a number ratio of 1:1:1, and oxygen (O) in the conventional-IGZO oxide satisfies a stoichiometric number ratio for indium (In), gallium (Ga), and zinc (Zn).

In an embodiment, the oxide semiconductor may include indium (In), gallium (Ga), zinc (Zn), oxygen (O), and inevitable impurities.

In an embodiment, Formula 1 may be represented by Formula 1-1:

$$Ef ( Con - IGZO ) - 0.1 eV < Ef < Ef ( C on - IGZO ) + 0.15 eV \qquad [ Formula 1 - 1 ]$$

wherein, in Formula 1-1, Ef and Ef (Con-IGZO) may be the same as defined in Formula 1.

In an embodiment, the oxide semiconductor may include indium (In), tin (Sn), gallium (Ga), zinc (Zn), oxygen (O), and inevitable impurities.

In an embodiment, Formula 1 may be represented by Formula 1-2:

$$Ef ( Con - IGZO ) - 0.1 eV < Ef < Ef ( Con - IGZO ) + 0.21 eV \qquad [ Formula 1 - 2 ]$$

wherein, in Formula 1-2, Ef and Ef (Con-IGZO) may be the same as defined in Formula 1.

In an embodiment, the oxide semiconductor may not include indium (In).

In an embodiment, the oxide semiconductor may include silver (Ag), gallium (Ga), zinc (Zn), oxygen (O), and inevitable impurities.

In an embodiment, the optimal composition ratio may further satisfy Formulas A1 and A2

$$0 < {N}_{A g} / ( {N}_{A g} + {N}_{G a} + {N}_{Zn} ) < 0.1 \qquad [ Formula A 1 ]$$

$$0.8 < {N}_{Ga} / {N}_{Zn} < 0.9 \qquad [ Formula A 2 ]$$

wherein, in Formulas A1 and A2, N_{Ag} may be a number of silver (Ag) atoms included in the oxide semiconductor, N_{Ga} may be a number of gallium (Ga) atoms included in the oxide semiconductor, and N_{Zn} may be a number of zinc (Zn) atoms included in the oxide semiconductor.

In an embodiment, the oxide semiconductor may include silver (Ag), magnesium (Mg), zinc (Zn), oxygen (O), and inevitable impurities.

In an embodiment, the optimal composition ratio may further satisfy Formulas B1 and B2:

$$\begin{matrix}0 < {N}_{A g} / ( {N}_{A g} + {N}_{G a} + {N}_{Zn} ) < 0.1 & [ Formula B 1 ]\end{matrix}$$

$$0.5 < {N}_{M g} / {N}_{Zn} < 0.7 \qquad [ Formula B 2 ]$$

wherein, in Formulas B1 and B2, N_{Ag} may be a number of silver (Ag) atoms included in the oxide semiconductor, N_{Mg} may be a number of magnesium (Mg) atoms included in the oxide semiconductor, and N_{Zn} may be a number of zinc (Zn) atoms included in the oxide semiconductor.

In an embodiment, the oxide semiconductor may include indium (In); at least one element (X) selected from the group consisting of gallium (Ga), zinc (Zn), tin (Sn), silver (Ag), aluminum (Al), cadmium (Cd), magnesium (Mg), antimony (Sb), silicon (Si), titanium (Ti), and zirconium (Zr); oxygen (O); and inevitable impurities.

In an embodiment, the optimal composition ratio may further satisfy Formula C:

$$0.55 < {N}_{In} / ( {N}_{In} + {N}_{X} ) < 0.9 \qquad [ Formula C ]$$

wherein, in Formula C, N_{In} may be a number of indium (In) atoms included in the oxide semiconductor, and N_{X} may be a number of atoms corresponding to the least one element (X) included in the oxide semiconductor.

In an embodiment, the oxide semiconductor may include indium (In); at least one element (Y) selected from the group consisting of gallium (Ga), zinc (Zn), tin (Sn), magnesium (Mg), and silicon (Si); oxygen (O); and inevitable impurities.

In an embodiment, the optimal composition ratio may further satisfy Formula D:

$$0.7 < {N}_{In} / ( {N}_{In} + {N}_{Y} ) < 0.9 \qquad [ Formula D ]$$

wherein, in Formula D, N_{In} may be a number of indium (In) atoms included in the oxide semiconductor, and N_{Y} may be a number of atoms corresponding to the at least one element (Y) included in the oxide semiconductor.

In an embodiment, the oxide semiconductor may include indium (In); at least one element (Z) selected from the group consisting of gallium (Ga), aluminum (Al), and tin (Sn); oxygen (O); and inevitable impurities.

In an embodiment, the optimal composition ratio may further satisfy Formula E:

$$0.85 < {N}_{In} / ( {N}_{In} + {N}_{Z} ) < 0.9 \qquad [ Formula E ]$$

wherein, in Formula E, N_{In} may be a number of indium (In) atoms included in the oxide semiconductor, and N_{Z} may be a number of atoms corresponding to the at least one element (Z) included in the oxide semiconductor.

An oxide semiconductor in accordance with embodiments of the disclosure may include at least one element selected from the group consisting of indium (In), gallium (Ga), zinc (Zn), tin (Sn), silver (Ag), aluminum (Al), cadmium (Cd), magnesium (Mg), antimony (Sb), silicon (Si), titanium (Ti), and zirconium (Zr); oxygen (O); and inevitable impurities, wherein the oxide semiconductor has an optimal composition ratio selected using the simulation method in accordance with the embodiments of the disclosure.

An electronic device in accordance with embodiments of the disclosure may include a display device to display an image. The display device may include an oxide semiconductor. The oxide semiconductor may include at least one element selected from the group consisting of indium (In), gallium (Ga), zinc (Zn), tin (Sn), silver (Ag), aluminum (Al), cadmium (Cd), magnesium (Mg), antimony (Sb), silicon (Si), titanium (Ti), and zirconium (Zr); oxygen (O); and inevitable impurities, wherein the oxide semiconductor has an optimal composition ratio selected using the simulation method in accordance with the embodiments of the disclosure.

### BRIEF DESCRIPTION OF THE DRAWINGS

FIG. 1 is a block diagram of an electronic device according to an embodiment;

FIG. 2 shows schematic views of various embodiments of an electronic device;

FIG. 3 is a schematic diagram illustrating a correlation between measured values of X1 and Vth for composition ratios C1 to C4;

FIG. 4 is a schematic diagram illustrating a correlation between measured values of X2 and Ion for the composition ratios C1 to C4; and

FIG. 5 is a schematic diagram illustrating a correlation between measured values of Vth and Ion for the composition ratios C1 to C4.

### DETAILED DESCRIPTION OF THE EMBODIMENTS

In the following description, for the purposes of explanation, numerous specific details are set forth in order to provide a thorough understanding of various embodiments or implementations of the disclosure. As used herein “embodiments” and “implementations” are interchangeable words that are non-limiting examples of devices or methods disclosed herein. It is apparent, however, that various embodiments may be practiced without these specific details or with one or more equivalent arrangements. Here, various embodiments do not have to be exclusive nor limit the disclosure. For example, specific shapes, configurations, and characteristics of an embodiment may be used or implemented in another embodiment.

Unless otherwise specified, the illustrated embodiments are to be understood as providing features of the disclosure. Therefore, unless otherwise specified, the features, components, modules, layers, films, panels, regions, and/or aspects, etc., (hereinafter individually or collectively referred to as “elements”), of the various embodiments may be otherwise combined, separated, interchanged, and/or rearranged without departing from the disclosure.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals and/or reference characters denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the X-axis, the Y-axis, and the Z-axis are not limited to three axes of a rectangular coordinate system, such as the x, y, and z axes, and may be interpreted in a broader sense. For example, the X-axis, the Y-axis, and the Z-axis may be perpendicular to one another, or may be different directions that are not perpendicular to one another.

For the purposes of this disclosure, “at least one of A and B” may be construed as A only, B only, or any combination of A and B. Also, “at least one of X, Y, and Z” and “at least one selected from the group consisting of X, Y, and Z” may be construed as X only, Y only, Z only, or any combination of two or more of X, Y, and Z. As used herein, the term “and/or” includes any and all combinations of one or more of the associated listed items.

Although the terms “first,” “second,” etc., may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, parts, and/or modules. Those skilled in the art will appreciate that these blocks, parts, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, parts, and/or modules being implemented by microprocessors or other similar hardware, they may be programmed and controlled using software (e.g., microcode) to perform various functions discussed herein and may optionally be driven by firmware and/or software. It is also contemplated that each block, part, and/or module may be implemented by dedicated hardware, or as a combination of dedicated hardware to perform some functions and a processor (e.g., one or more programmed microprocessors and associated circuitry) to perform other functions. Also, each block, part, and/or module of some embodiments may be physically separated into two or more interacting and discrete blocks, parts, and/or modules without departing from the scope of the disclosure. Further, the blocks, parts, and/or modules of some embodiments may be physically combined into more complex blocks, parts, and/or modules without departing from the scope of the disclosure.

Unless otherwise defined or implied herein, all terms (including technical and scientific terms) used herein have the same meaning as commonly understood by those skilled in the art to which this disclosure pertains. It will be further understood that terms, such as those defined in commonly used dictionaries, should be interpreted as having a meaning that is consistent with their meaning in the context of the relevant art and the disclosure, and should not be interpreted in an ideal or excessively formal sense unless clearly so defined herein.

The disclosure relates to a simulation method for selecting an optimal composition ratio of an oxide semiconductor.

### <Oxide Semiconductor>

An oxide semiconductor of the disclosure may include at least two elements selected from the group consisting of indium (In), gallium (Ga), zinc (Zn), tin (Sn), silver (Ag), aluminum (Al), cadmium (Cd), magnesium (Mg), antimony (Sb), silicon (Si), titanium (Ti), and zirconium (Zr); oxygen (O); and inevitable impurities.

In an embodiment, the oxide semiconductor may include indium (In).

For example, the oxide semiconductor may include indium (In), gallium (Ga), zinc (Zn), oxygen (O), and inevitable impurities. The oxide semiconductor may be referred to as an IGZO oxide semiconductor.

As another example, the oxide semiconductor may include indium (In), tin (Sn), gallium (Ga), zinc (Zn), oxygen (O), and inevitable impurities. The oxide semiconductor may be referred to as an ITGZO oxide semiconductor.

In an embodiment, the oxide semiconductor may not include indium (In).

For example, the oxide semiconductor may include silver (Ag), gallium (Ga), zinc (Zn), oxygen (O), and inevitable impurities.

As another example, the oxide semiconductor may include silver (Ag), magnesium (Mg), zinc (Zn), oxygen (O), and inevitable impurities.

As such, the oxide semiconductor may include various combinations of the above-described elements. The above-described elements may exist in a specific composition ratio in the oxide semiconductor, and characteristics (e.g., mobility, threshold voltage (Vth), on current (Ion), etc.) of the transistor including the oxide semiconductor may be determined by the specific composition ratio.

Therefore, it is desirable to select an optimal composition ratio of the elements that constitute the oxide semiconductor so that the transistor including the oxide semiconductor has optimal characteristics.

### <Simulation Method>

A simulation method of the disclosure includes setting a simulation target composition ratio set including various composition ratios of the above-described elements constituting the oxide semiconductor, checking whether the oxide semiconductor satisfies Formulas 1, 2, and 3 below for each of the various composition ratios included in the simulation target composition ratio set, and selecting a composition ratio satisfying Formulas 1, 2, and 3 below as an optimal composition ratio.

$$Ef ( Con - IGZO ) - 0.1 eV < Ef < Ef ( Con - IGZO ) + 0.3 eV \qquad [ Formula 1 ]$$

In Formula 1 above, Ef is formation energy of the oxide semiconductor calculated based on density functional theory (DFT). Any of various programs may be used for calculations based on the DFT without any limitation.

In Formula 1 above, Ef may be represented by Formula 1A below.

$$Ef = ( Etot ( sys ) - SUM ( Etot ( atom ) ) / {N}_{O} \qquad [ Formula 1 A ]$$

In Formula 1A above, Etot(sys) is a value obtained by calculating total energy of the oxide semiconductor based on the DFT, SUM(Etot(atom)) is the sum of energies of individual elements constituting the oxide semiconductor, and N_{O} represents the stoichiometric number of oxygen (O) constituting the oxide semiconductor.

In Formula 1 above, Ef (Con-IGZO) is formation energy of the conventional-IGZO oxide calculated based on the DFT. The conventional-IGZO oxide includes indium (In), gallium (Ga), zinc (Zn), and oxygen (O). In the conventional-IGZO oxide, indium (In):gallium (Ga):zinc (Zn) satisfies a number ratio of 1:1:1, and oxygen (O) satisfies a stoichiometric number ratio for indium (In), gallium (Ga), and zinc (Zn).

Formula 1 above may limit the bonding stability of the oxide semiconductor represented by Ef to a certain range with respect to the bonding stability of the conventional-IGZO oxide represented by Ef (Con-IGZO). In case that Ef satisfies the range described in Formula 1 above, the stability of the oxide semiconductor may be improved and the probability of oxygen vacancy may be reduced.

$$[ 2 eV < Eg < 3.5 eV \qquad [ Formula 2 ]$$

In Formula 2 above, Eg is bandgap energy of the oxide semiconductor calculated based on the DFT. More specifically, the calculation of Eg based on the DFT may be performed by an extension of a many-body theory derived from the Schrödinger Equation. Any of various programs may be used for calculations based on the DFT without any limitation.

In case that Eg satisfies the range described in Formula 2 above, the optical reliability of the oxide semiconductor may be improved, and the oxide semiconductor may be prevented from having characteristics of a conductor or an insulator.

$$( 100 \cdot {( ISWOc )}^{- 2} \cdot exp ( Ef ) > 3 \qquad [ Formula 3 ]$$

In Formula 3 above, ISWOc is an Inverse State Weighted Overlap of Conduction band parameter of the oxide semiconductor. The ISWOc may be a parameter defining the degree of overlap of orbitals of elements included in the oxide semiconductor in a conduction band. The ISWOc may be calculated by a method known in A. de Jamblinne de Meux et al., Method to quantify the delocalization of electronic states in amorphous semiconductors and its application to assessing charge carrier mobility of p-type amorphous oxide semiconductors, Physical Review B 97, 045208 (2018).

The left side of Formula 3 above correlates the carrier density of the oxide semiconductor and the intrinsic mobility of the oxide semiconductor, and may represent the mobility index of the oxide semiconductor. In case that the mobility index of the oxide semiconductor satisfies the range described in Formula 3 above, the oxide semiconductor may have excellent mobility.

In an embodiment, the checking of whether the oxide semiconductor satisfies Formulas 1 to 3 for each of the various composition ratios included in the simulation target composition ratio set may be performed by a machine-learning AI model. The inspection time for checking all of the various composition ratios included in the simulation target composition ratio set may be shortened, and data on the optimal composition ratio at which the oxide semiconductor is expected to exhibit optimal device characteristics may be readily obtained.

In an embodiment, the oxide semiconductor may include indium (In), gallium (Ga), zinc (Zn), oxygen (O), and inevitable impurities. Formula 1 of the simulation method of the disclosure may be modified in consideration of consistency with actual measurements of the mobility and threshold voltage of the thin film transistor to which the oxide semiconductor is applied. More specifically, Formula 1 above may be represented by Formula 1-1 below.

$$Ef ( Con - IGZO ) - 0.1 eV < Ef < Ef ( C on - IGZO ) + 0.15 eV \qquad [ Formula 1 - 1 ]$$

In Formula 1-1 above, Ef and Ef (Con-IGZO) are the same as defined in Formula 1 above.

In an embodiment, the oxide semiconductor may include indium (In), tin (Sn), gallium (Ga), zinc (Zn), oxygen (O), and inevitable impurities. Formula 1 of the simulation method of the disclosure may be modified in consideration of consistency with actual measurements of the mobility and threshold voltage of the thin film transistor to which the oxide semiconductor is applied. More specifically, Formula 1 above may be represented by Formula 1-2 below.

$$Ef ( Con - IGZO ) - ( 0 , 1 eV < Ef < Ef ( C on - IGZO ) + 0.21 eV \qquad [ Formula 1 - 2 ]$$

In Formulas 1-2 below, Ef and Ef (Con-IGZO) may be the same as defined in Formula 1 above.

As such, Formula 1 above may be variously modified depending on the type of elements constituting the oxide semiconductor.

### <Optimal Composition Ratio of Oxide Semiconductor Selected by Simulation Method>

In an embodiment, the oxide semiconductor may be configured not to include indium (In).

For example, the oxide semiconductor may include silver (Ag), gallium (Ga), zinc (Zn), oxygen (O), and inevitable impurities. The optimal composition ratio selected from the simulation method of the disclosure may further satisfy Formulas A1 and A2 below.

$$0 < {N}_{Ag} / ( {N}_{Ag} + {N}_{G a} + {N}_{Zn} ) < 0.1 \qquad [ Formula A 1 ]$$

$$0.8 < {N}_{G a} / {N}_{Zn} < 0.9 \qquad [ Formula A 2 ]$$

In Formulas A1 and A2, N_{Ag} is the number of silver (Ag) atoms included in the oxide semiconductor, N_{Ga} is the number of gallium (Ga) atoms included in the oxide semiconductor, and N_{Zn} is the number of zinc (Zn) atoms included in the oxide semiconductor.

As another example, the oxide semiconductor may include silver (Ag), magnesium (Mg), zinc (Zn), oxygen (O), and inevitable impurities. The optimal composition ratio selected from the simulation method of the disclosure may further satisfy Formulas B1 and B2 below.

$$0 < {N}_{A g} / ( {N}_{A g} + {N}_{Mg} + {N}_{Zn} ) < 0.1 \qquad [ Formula B 1 ]$$

$$0.5 < {N}_{Mg} / {N}_{Zn} < 0.7 \qquad [ Formula B 2 ]$$

In Formulas B1 and B2 above, N_{Ag} is the number of silver (Ag) atoms included in the oxide semiconductor, N_{Mg} is the number of magnesium (Mg) atoms included in the oxide semiconductor, and N_{Zn} is the number of zinc (Zn) atoms included in the oxide semiconductor.

In an embodiment, the oxide semiconductor may include indium (In).

For example, the oxide semiconductor may include at least one element (X) selected from the group consisting of gallium (Ga), zinc (Zn), tin (Sn), silver (Ag), aluminum (Al), cadmium (Cd), magnesium (Mg), antimony (Sb), silicon (Si), titanium (Ti), and zirconium (Zr), indium (In); oxygen (O); and inevitable impurities. The optimal composition ratio selected from the simulation method of the disclosure may further satisfy Formula C below.

$$0.55 < {N}_{In} / ( {N}_{In} + {N}_{X} ) < 0.9 \qquad [ Formula C ]$$

In Formula C above, N_{In} is the number of indium (In) atoms included in the oxide semiconductor, and N_{X} is the number of atoms corresponding to the at least one element (X) included in the oxide semiconductor.

As an example, the oxide semiconductor may include at least one element (Y) selected from the group consisting of gallium (Ga), zinc (Zn), tin (Sn), magnesium (Mg), and silicon (Si), indium (In); oxygen (O); and inevitable impurities. The optimal composition ratio selected from the simulation method of the disclosure may further satisfy Formula D below.

$$0.7 < {N}_{In} / ( {N}_{In} + {N}_{Y} ) < 0.9 \qquad [ Formula D ]$$

In Formula D above, N_{In} is the number of indium (In) atoms included in the oxide semiconductor, and N_{Y} is the number of atoms corresponding to the at least one element (Y) included in the oxide semiconductor.

As another example, the oxide semiconductor may include at least one element (Z) selected from the group consisting of gallium (Ga), aluminum (Al), and tin (Sn), indium (In); oxygen (O); and inevitable impurities. The optimal composition ratio selected from the simulation method of the disclosure may further satisfy Formula E below.

$$0.85 < {N}_{In} / ( {N}_{In} + {N}_{Z} ) < 0.9 \qquad [ Formula E ]$$

In Formula E above, N_{In} is the number of indium (In) atoms included in the oxide semiconductor, and N_{Z} is the number of atoms corresponding to the at least one element (Z) included in the oxide semiconductor.

As such, according to the simulation method of the disclosure, an optimal composition ratio may be derived depending on the type of elements included in the oxide semiconductor.

### <Electronic Device>

In an embodiment, an electronic device includes a display device and may further include other modules or devices having additional functions in addition to the display device. The display device may include above described oxide semiconductor, and optimal composition ratio of the above described oxide semiconductor may be selected by the simulation method of embodiments of the disclosure.

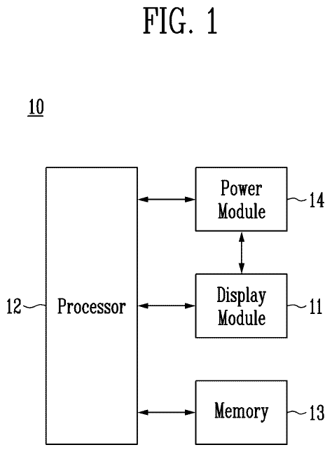

FIG. 1 is a block diagram of an electronic device according to an embodiment. Referring to FIG. 1, the electronic device 10 may include a display module 11, a processor 12, a memory 13, and a power module 14.

The processor 12 may include at least one of a central processing unit (CPU), an application processor (AP), a graphic processing unit (GPU), a communication processor (CP), an image signal processor (ISP), and a controller.

The memory 13 may store data and/or information used to operate the processor 12 or the display module 11. When the processor 12 executes an application stored in the memory 13, image data signals and/or input control signals may be transferred to the display module 11. The display module 11 may process the provided signals and output image information on a display screen.

The power module 14 may include a power supply module, such as a power adapter or a battery device, and a power conversion module. The power conversion module converts power supplied by the power supply module and generates power to operate the electronic device 10.

At least one of the above-described components of the electronic device 10 may be included in the display device according to embodiments as described above. In addition, in terms of functionality, some of the individual modules included in one module may be included in the display device and others may be provided separately from the display device. For example, the display module 11 is included in the display device, whereas the processor 12, the memory 13, and the power module 14 are not included in the display device and are instead provided separately in the electronic device 10.

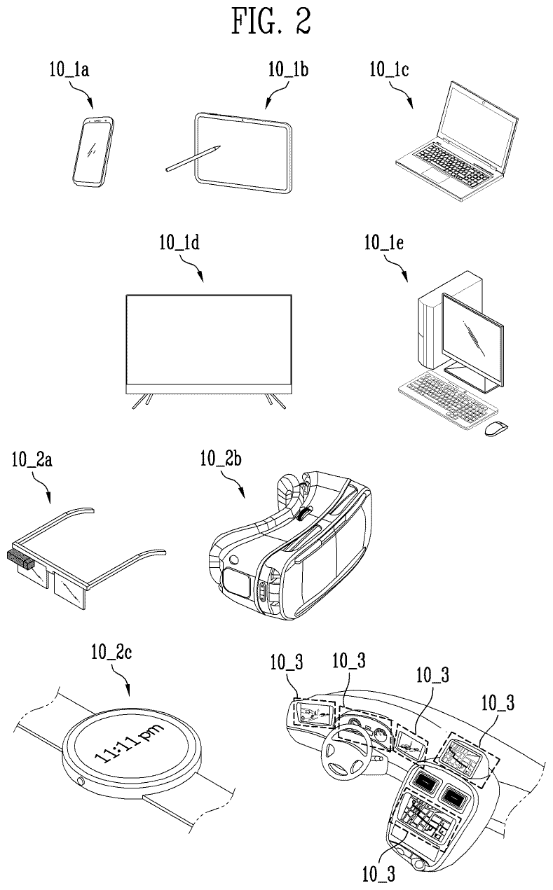

FIG. 2 shows schematic views of various embodiments of an electronic device.

Referring to FIG. 2, various types of electronic devices to which embodiments of a display device are applied may include an electronic device to display images such as a smartphone 10_1a, a tablet PC 10_1b, a laptop computer 10_1c, a television (TV) 10_1d, and a desktop monitor 10_1e, a wearable electronic device including a display module such as smart glasses 10_2a, a head-mounted display (HMD) 10_2b, and a smart watch 10_2c, and an automotive electronic device 10_3 including a display module such as a center information display (CID) disposed at the instrument cluster, the center fascia, and the dashboard of a vehicle, and a room mirror display.

Hereinafter, embodiments of the disclosure will be described in detail so that those skilled in the art can readily carry out the disclosure. However, the disclosure may be implemented in various different forms and is not limited to the embodiments described herein.

The simulation method of the disclosure has been performed on the oxide semiconductor including indium (In), tin (Sn), gallium (Ga), zinc (Zn), oxygen (O), and unavoidable impurities, and the results thereof are shown in Table 1 below.

TABLE 1

| | Composition ratio (number ratio) | | | | Simulation result | |
|---|---|---|---|---|---|---|
| | In | Sn | Ga | Zn | X1 | X2 |
| C1 | 60 | 2.7 | 14.3 | 23 | 0.25 eV | 3.27 |
| C2 | 60 | 2.7 | 20.3 | 17 | 0.22 eV | 3.17 |
| C3 | 60 | 2.7 | 22 | 15.3 | 0.20 eV | 3.12 |
| C4 | 60 | 2.7 | 24 | 13.3 | 0.13 eV | 2.94 |

In Table 1 above, X1 is a value of Ef-Ef (Con-IGZO) for each composition and was calculated based on Formula 1 described above. In Table 1 above, X2 is a value of 100·(ISWOc)-2·exp (Ef) for each composition and was calculated based on Formula 3 described above.

In Table 1 above, oxide semiconductors including composition ratios C1 to C4 satisfied the condition of Formula 2 described above.

Thin film transistors including the oxide semiconductors satisfying the composition ratios C1 to C4 were manufactured, and the on current (Ion) and threshold voltage (Vth) were measured under conditions of a gate-source voltage of 15 V and a drain-source voltage of 5.1 V. Results thereof are shown in Table 2 below.

TABLE 2

| | Composition ratio (number ratio) | | | | Measurement result | | |
|---|---|---|---|---|---|---|---|
| | In | Sn | Ga | Zn | Ion | | Vth |
| C1 | 60 | 2.7 | 14.3 | 23 | 139.22 | A | −6.31 V |
| C2 | 60 | 2.7 | 20.3 | 17 | 111.68 | A | −4.28 V |
| C3 | 60 | 2.7 | 22 | 15.3 | 101.87 | A | −3.62 V |
| C4 | 60 | 2.7 | 24 | 13.3 | 81.15 | A | −3.01 V |

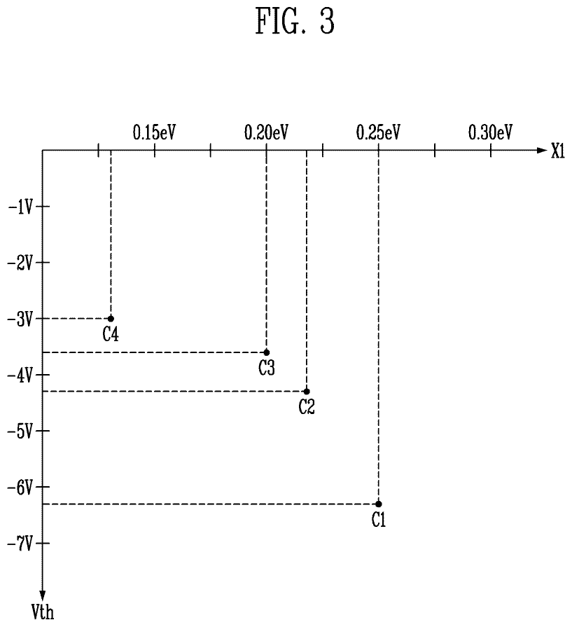

FIG. 3 is a schematic diagram illustrating a correlation between measured values of X1 and Vth for composition ratios C1 to C4.

Referring to FIG. 3, as X1 increases, the measured Vth value tends to increase in the negative direction (i.e., increased magnitude of negative voltage for Vth), and from this, it may be confirmed that the consistency between X1 calculated by the simulation method of the disclosure and the measured Vth value is excellent, and thus Vth characteristics of the device may be predicted based on Formula 1 of the disclosure.

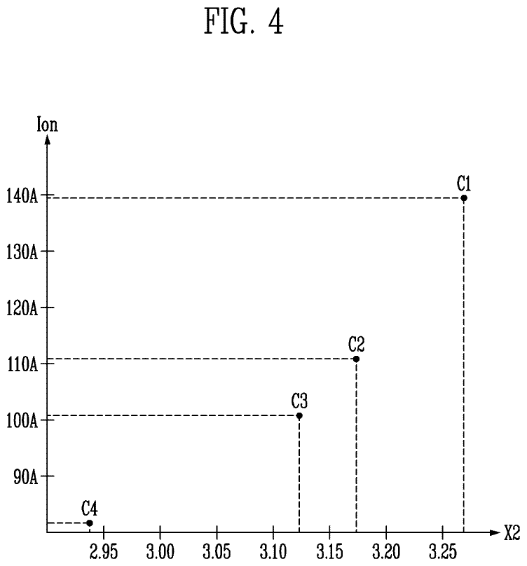

FIG. 4 is a schematic diagram illustrating a correlation between measured values of X2 and Ion in the composition ratios C1 to C4.

Referring to FIG. 4, as X2 increases, the measured Ion value tends to increase, and from this, it may be confirmed that the consistency between the X2 calculated by the simulation method of the disclosure and the measured Ion value is excellent, and thus Ion characteristics of the device may be predicted based on Formula 3 of the disclosure.

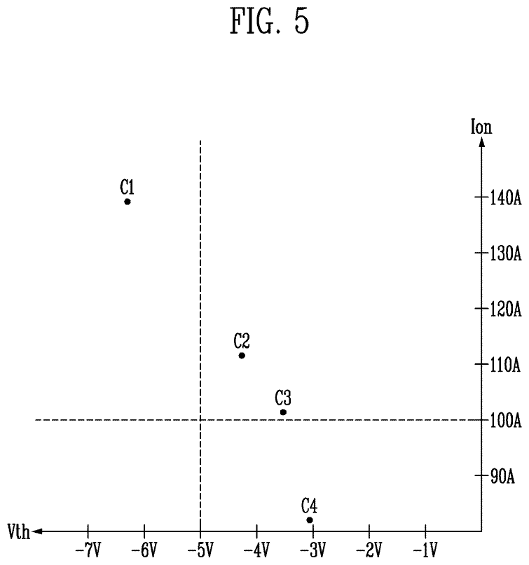

FIG. 5 is a schematic diagram illustrating a correlation between measured values of Vth and Ion in the composition ratios C1 to C4.

Referring to FIG. 5, it may be confirmed that both the Vth measured value and the Ion measured value are excellent in the composition ratios C2 and C3 satisfying Formulas 1 to 3 in the simulation method of the disclosure.

In contrast, in the simulation method of the disclosure, it may be confirmed that the measured Vth value is excessively low for the composition ratio C1 that does not satisfy Formula 1, resulting in poor device characteristics.

In the simulation method of the disclosure, it may be confirmed that the measured Ion value is excessively low for the composition ratio C4 that does not satisfy Formula 3, resulting in poor device characteristics.

In accordance with embodiments of the disclosure, there may be provided an optimal composition ratio that allows an oxide semiconductor including various elements to exhibit optimal device characteristics.

Although the disclosure has been described above with reference to embodiments, those skilled in the art will understand that various modifications and changes may be made thereto without departing from the scope of the disclosure described in the appended claims.

What is claimed is:

1. A simulation method for selecting an optimal composition ratio of an oxide semiconductor that includes at least two elements selected from the group consisting of indium (In), gallium (Ga), zinc (Zn), tin (Sn), silver (Ag), aluminum (Al), cadmium (Cd), magnesium (Mg), antimony (Sb), silicon (Si), titanium (Ti), and zirconium (Zr); oxygen (O); and inevitable impurities, the simulation method comprising:
setting a simulation target composition ratio set including various composition ratios of elements constituting the oxide semiconductor;
checking whether the oxide semiconductor satisfies Formulas 1, 2, and 3 below for each of the various composition ratios included in the simulation target composition ratio set; and
selecting a composition ratio satisfying Formulas 1, 2, and 3 below as an optimal composition ratio:
$$Ef ( Con - IGZO ) - 0.1 eV < Ef < Ef ( Con - IGZO + 0.3 eV \qquad [ Formula 2 ]$$
$$2 eV < E g < 35 eV \qquad [ Formula 2 ]$$
$$100 \cdot {( ISWOc )}^{- 2} \cdot exp ( Ef ) > 3 \qquad [ Formula 3 ]$$
wherein, in Formulas 1, 2, and 3,
Ef is formation energy of the oxide semiconductor calculated based on density functional theory (DFT),
Eg is bandgap energy of the oxide semiconductor calculated based on the DFT,
ISWOc is an Inverse State Weighted Overlap of Conduction band parameter of the oxide semiconductor,
Ef (Con-IGZO) is formation energy of conventional-IGZO oxide,
the conventional-IGZO oxide includes indium (In), gallium (Ga), zinc (Zn), and oxygen (O),
indium (In): gallium (Ga): zinc (Zn) in the conventional-IGZO oxide satisfies a number ratio of 1:1:1, and
oxygen (O) in the conventional-IGZO oxide satisfies a stoichiometric number ratio for indium (In), gallium (Ga), and zinc (Zn).

2. The simulation method of claim 1, wherein the oxide semiconductor includes indium (In), gallium (Ga), zinc (Zn), oxygen (O), and inevitable impurities.

3. The simulation method of claim 2, wherein Formula 1 is represented by Formula 1-1:
$$Ef ( Con - IGZO ) - 0.1 eV < Ef < Ef ( C on - IGZO ) + 0.15 eV \qquad [ Formula 1 - 1 ]$$
wherein, in Formula 1-1, Ef and Ef (Con-IGZO) are the same as defined in Formula 1.

4. The simulation method of claim 1, wherein the oxide semiconductor includes indium (In), tin (Sn), gallium (Ga), zinc (Zn), oxygen (O), and inevitable impurities.

5. The simulation method of claim 4, wherein Formula 1 is represented by Formula 1-2:
$$Ef ( Con - IGZO ) - 0.1 eV < Ef < Ef ( C on - IGZO ) + 0.21 eV \qquad [ Formula 1 - 2 ]$$
wherein, in Formula 1-2, Ef and Ef (Con-IGZO) are the same as defined in Formula 1.

6. The simulation method of claim 1, wherein the oxide semiconductor does not include indium (In).

7. The simulation method of claim 6, wherein the oxide semiconductor includes silver (Ag), gallium (Ga), zinc (Zn), oxygen (O), and inevitable impurities.

8. The simulation method of claim 7, wherein the optimal composition ratio further satisfies Formulas A1 and A2:
$$0 < {N}_{A g} / ( {N}_{A g} + {N}_{G a} + {N}_{Zn} ) < 0.1 \qquad [ Formula A 1 ]$$
$$0.8 < {N}_{G a} / {N}_{Zn} < 0.9 \qquad [ Formula A 2 ]$$
wherein, in Formulas A1 and A2,
N_{Ag} is a number of silver (Ag) atoms included in the oxide semiconductor,
N_{Ga} is a number of gallium (Ga) atoms included in the oxide semiconductor, and
N_{Zn} is a number of zinc (Zn) atoms included in the oxide semiconductor.

9. The simulation method of claim 6, wherein the oxide semiconductor includes silver (Ag), magnesium (Mg), zinc (Zn), oxygen (O), and inevitable impurities.

10. The simulation method of claim 9, wherein the optimal composition ratio further satisfies Formulas B1 and B2:
$$0 < {N}_{A g} / ( {N}_{A g} + {N}_{Mg} + {N}_{Zn} ) < 0.1 \qquad [ Formula B 1 ]$$
$$0.5 < {N}_{Mg} / {N}_{Zn} < 0.7 \qquad [ Formula B 2 ]$$
wherein, in Formulas B1 and B2,
N_{Ag} is a number of silver (Ag) atoms included in the oxide semiconductor,
N_{Mg} is a number of magnesium (Mg) atoms included in the oxide semiconductor, and
N_{Zn} is a number of zinc (Zn) atoms included in the oxide semiconductor.

11. The simulation method of claim 1, wherein the oxide semiconductor comprises:
indium (In);
at least one element (X) selected from the group consisting of gallium (Ga), zinc (Zn), tin (Sn), silver (Ag), aluminum (Al), cadmium (Cd), magnesium (Mg), antimony (Sb), silicon (Si), titanium (Ti), and zirconium (Zr);
oxygen (O); and
inevitable impurities.

12. The simulation method of claim 11, wherein the optimal composition ratio further satisfies Formula C:
$$0.55 < {N}_{In} / ( {N}_{In} + {N}_{X} ) < 0.9 \qquad [ Formula C ]$$
wherein, in Formula C,
N_{In} is a number of indium (In) atoms included in the oxide semiconductor, and
N_{X} is a number of atoms corresponding to the at least one element (X) included in the oxide semiconductor.

13. The simulation method of claim 1, wherein the oxide semiconductor comprises:
indium (In);
at least one element (Y) selected from the group consisting of gallium (Ga), zinc (Zn), tin (Sn), magnesium (Mg), and silicon (Si);
oxygen (O); and
inevitable impurities.

14. The simulation method of claim 13, wherein the optimal composition ratio further satisfies Formula D:
$$0.7 < {N}_{In} / ( {N}_{In} + {N}_{Y} ) < 0.9 \qquad [ Formula D ]$$
wherein, in Formula D,
N_{In} is a number of indium (In) atoms included in the oxide semiconductor, and
N_{Y} is a number of atoms corresponding to the at least one element (Y) included in the oxide semiconductor.

15. The simulation method of claim 1, wherein the oxide semiconductor comprises:
indium (In);
at least one element (Z) selected from the group consisting of gallium (Ga), aluminum (Al), and tin (Sn);
oxygen (O); and
inevitable impurities.

16. The simulation method of claim 15, wherein the optimal composition ratio further satisfies Formula E:
$$0.85 < {N}_{In} / ( {N}_{In} + {N}_{Z} ) < 0.9 \qquad [ Formula E ]$$
wherein, in Formula E,
N_{In} is a number of indium (In) atoms included in the oxide semiconductor, and
N_{Z} is a number of atoms corresponding to the at least one element (Z) included in the oxide semiconductor.

17. An oxide semiconductor comprising:
at least one element selected from the group consisting of indium (In), gallium (Ga), zinc (Zn), tin (Sn), silver (Ag), aluminum (Al), cadmium (Cd), magnesium (Mg), antimony (Sb), silicon (Si), titanium (Ti), and zirconium (Zr);
oxygen (O); and
inevitable impurities,
wherein the oxide semiconductor has an optimal composition ratio selected using the simulation method of claim 1.

18. An electronic device comprising:
a display device that displays an image, the display device including an oxide semiconductor,
wherein the oxide semiconductor comprises:
at least one element selected from the group consisting of indium (In), gallium (Ga), zinc (Zn), tin (Sn), silver (Ag), aluminum (Al), cadmium (Cd), magnesium (Mg), antimony (Sb), silicon (Si), titanium (Ti), and zirconium (Zr);
oxygen (O); and
inevitable impurities,
wherein the oxide semiconductor has an optimal composition ratio selected using the simulation method of claim 1.
